(19) United States
(12) Patent Application Publication
(10) Pub. No.: US 2008/0254015 A1
(43) Pub. Date: Oct. 16, 2008

(54) Gel Composition of Citrus Complex Carbohydrates Cross-Linked with Cellulose Derivate

(75) Inventors: Yousef Samih Taktak, Matlock (GB); Michael William Gill, Cheshire (GB)

Correspondence Address: MCHALE & SLAVIN, P.A., 2855 PGA BLVD, PALM BEACH GARDENS, FL, 33410, US

(73) Assignee: AVANTICARE LTD, Sheffield (GB)

(21) Appl. No.: 10/597,189

(22) PCT Filed: Jan. 12, 2005
(86) PCT No.: PCT/GB2005/000076

§ 371 Date: Jun. 27, 2008

(30) Foreign Application Priority Data
Jan. 14, 2004 (GB) 0400775.3

(51) Int. Cl. A61K 38/43; A61K 31/717; A61K 36/886; A61P 31/02

(52) U.S. Cl. 424/94.1; 514/57; 424/744

(57) ABSTRACT

A gel for application to a wound, comprising a mixture of citrus complex carbohydrates, a cellulose derivative, a polyol component and water, wherein said citrus complex carbohydrates are cross-linked to said cellulose derivative by an ionic cross-linking agent.

The present invention relates to a gel composition, and more particularly to a gel composition for topical application.

It is known that the presence of moisture at a wound site helps promote healing of the wound. However, the accumulation of wound exudate or excessive moisture at the wound site can cause maceration of the skin adjacent the wound and can also promote the growth of bacteria and other organisms which may delay healing of the wound and also lead to infection.

In WO92/16245 there is described a wound dressing containing a water-insoluble, water swellable cross-linked cellulose derivative, water and a polyol component and comprising a gel wherein the cellulose derivative comprises less than 10% by weight of the gel. The adherency of such known hydrogels tends to be less than ideal, and they also have a tendency to disintegrate in the wound and to cause maceration of the skin around the wound.

In WO95/17166 there is described a wound hydrating gel comprising a) a hydrocolloid mixture of carboxymethylcellulose and sodium/calcium alginate, and b) a preservative system.

In GB2322864 there is described a gel for application to the human or animal body, comprising a first carboxy-polysaccharide, a second carboxy-polysaccharide and multivalent ions providing ionic cross-links between said polysaccharides.

The present invention provides a gel for topical application to a wound that can minimise the accumulation of excessive moisture at a wound site whilst maintaining a level of moisture at the site which promotes wound healing.

In a first aspect, the present invention provides a gel for topical application to a wound, the gel comprising a mixture of citrus complex carbohydrates, a cellulose derivative, a polyol component and water, wherein said citrus complex carbohydrates are cross-linked to said cellulose derivative by an ionic cross-linking agent.

By a gel in this specification is meant a three-dimensional network of a super absorbent polymer which interacts with aqueous solutions by swelling and retains a significant proportion of water within its structure. Preferably the polymer is fully saturated with water. By a super absorbent polymer is meant a polymer capable of absorbing at least ten times its own weight of water.

Preferably the gel is an amorphous gel, that is to say, it is unstructured and capable of flowing under pressure. Preferably the gel has sufficient structural integrity, however, that it can be peeled from a surface, for example, from the surface of a wound. Preferably the gel has a viscosity of greater than 50,000 cp.

Preferably, the mixture of citrus complex carbohydrates is extracted from citrus fruit peel, for example, from lemon, lime, orange, or grapefruit peel, or from a mixture thereof. The extract contains a mixture of sugars, and a high proportion of pectin. Pectin (E440) is an acidic structural polysaccharide with a complex structure. Preparations consist of substructural entities that depend on their source and extraction methodology. The majority of the structure consists of homopolymeric partially methylated poly-α-(1-4)-D-galacturonic acid residues but there are substantial non-gelling areas of alternating α-(1-2)-L-rhamnosyl-α-(1-4)-D-galacturonosyl sections containing branch-points with mostly neutral side chains (1-20 residues) of mainly L-arabinose and D-galactose (rhamnogalacturonan I). Pectins may also contain rhamnogalacturonan II sidechains containing other residues such as D-xylose, L-fucose, D-glucuronic acid, D-apiose, 3-deoxy-D-manno-2-octulosonic acid (Kdo) and 3-deoxy-D-lyxo-2-heptulosonic acid (Dha) attached to poly-α-(1-4)-D-galacturonic acid regions Generally, pectins do not possess exact structures.

Preferably the citrus complex carbohydrate is extracted from the citrus peel by leaching using an aqueous medium, preferably hot acidified water. The citrus complex carbohydrates are obtainable, for example, from the peel of lemon, lime, orange and grapefruit. The extracted carbohydrates can be clarified, for example, by centrifugation followed by a plurality of filtrations. The clarified carbohydrates can be further purified, for example, by precipitation of the carbohydrate from the clarified liquid extract. Precipitation may be carried out with alcohol from a concentrated complex carbohydrate solution or with aluminium from a diluted carbohydrate solution.

The citrus complex carbohydrates used in the invention can be either low or high ester carbohydrates, or both, but low ester carbohydrates are preferred (less than 40% esterified). Low-ester complex carbohydrates can be obtained by controlled de-esterification of high ester complex carbohydrates under either acid or alkaline conditions.

Preferably, the gel comprises from 0.01 to 10% by weight of the citrus complex carbohydrate, based on the weight of the gel, more preferably about 2.8% by weight, based on the weight of the gel.

Suitable cross-linked cellulose derivatives for use in the present invention include, for example, hydroxy lower alkyl cellulose derivatives wherein the alkyl group comprises from 1 to 6 carbon atoms, for example, hydroxyethylcellulose and hydroxypropylcellulose; and the carboxy-celluloses, for example, carboxymethylhydroxyethyl-cellulose and carboxymethylcellulose. Preferably, the cellulose derivative is a carboxymethyl cellulose derivative.

Ionic cellulose derivatives derived from carboxy celluloses are particularly preferred. Carboxymethyl cellulose in the form of its sodium salt is a preferred cellulose derivative. It is readily available and is the cheapest form of carboxymethylcellulose. However, other alkali metal and alkaline earth metal salts may also be used, for example, lithium, potassium and calcium.

Carboxymethylcellulose may be prepared, for example, by the reaction of cellulose with the sodium salt of chloroacetic acid in an aqueous alkaline organic slurry. Thus cellulose is steeped in sodium hydroxide solution and the alkali cellulose is treated under controlled conditions with sodium monochloroacetate to form the sodium salt of carboxymethyl cellulose and sodium chloride.

Preferably, the gel comprises 0.01% to 10% by weight of the cellulose derivative, based on the weight of the gel, more preferably about 3.7% by weight, based on the weight of the gel.

Polyols for use in the present invention are preferably water miscible and, more preferably liquid at room temperature. Polyols suitable for use in the present invention include dihydroxyalkanes, for example glycols having from 2 to 6 carbon atoms, for example, 1,2-dihydroxypropane, 2,3-dihydroxybutane and 3,4-dihydroxyhexane, 2,5-dihydroxyalkane, and diethylene and triethylene glycols.

1,2-dihydroxypropane (propylene glycol) is the preferred dihydroxyalkane for use in the gel of the present invention.

Polyhydroxyalkanes of the general formula:

CₙH(₂ₙ₊₂)Oₙ

in which n is a number from 3 to 6, are also suitable for use as polyols in the preparation of a gel of the invention and include, for example, glycerin, sorbitol, mannitol, adonite, ribite, dulcitol, erythritol and xylite.

Preferably, however, the polyol is a polyalkylene glycol. More preferably, the polyol is a water-soluble polyethylene glycol having a molecular weight in the range of from 200 to 600. A polypropylene glycol that can also be used is water-soluble and has a molecular weight of in the range of 200 to 450.

It is also possible to use a mixture of polyols.

The polyols act as humectants. They have the effect of reducing the partial vapour pressure of water so that the gel does not dry out. As a result, adhesion to the wounds or edges of the wounds is avoided, so that the gel may be removed without difficulty. Furthermore, they aid in the dispersion of the cellulose derivative in water during processing. They also render to the gel physical properties which makes it more easy to apply. It is also believed that they enhance the moisture penetration of necrotic tissue and thus speed up the debriding action. In addition, it is believed that they may act as biostatic agents, stopping the growth of micro-organisms.

Preferably, the gel comprises 0.1 to 30% by weight of the polyol component, based on the weight of the gel, more preferably about 14.4% by weight, based on the weight of the gel.

Preferably, the ionic cross-linking agent is a multivalent ion, more preferably a divalent ion, for example, a magnesium ion, or a calcium ion.

Preferably, the gel comprises 0.01 to 5% by weight of the ionic cross-linking agent, based on the weight of the gel, more preferably 0.9% by weight, based on the weight of the gel.

The gel of the present invention may, in addition to the aforesaid components, contain any additive and auxiliary substance (for example perfumes) customary for such compositions, as well as, in therapeutically active amounts, active substances(s) as wound treatment agents. Such active substances include, for example, antibacterial agents, antimycotics and local anaesthetics. Suitable antibacterial agents are sulphonamides, for example, suldacine and sulphatolamide, or antibiotics, for example penicillin and metronidazole. Suitable antimycotics are salicylic acid and derivatives thereof, for example salicylhydroxamic acid, salicylamide, miconacol and isoconacol. Suitable local anaesthetics are alkaloids, for example morphine, and esters of p-aminobenzoic acid, for example, the methyl ester and the ethyl ester.

Other active substances that can be included are an anti-fungal agent; an anti-inflammatory agent; an enzyme; and nutrients, for example, one or more vitamins, especially vitamin E; one or more amino acids; aloe vera; honey; or one or more trace metals.

The gel may also contain additional debriding agents e.g. enzymatic debriding agents and/or growth factors.

The pH of the gel should lie in a therapeutically desirable range. Preferably the pH should lie in the range of from pH 3 to 11, more preferably in the range between pH 5 and 6.

In a preferred embodiment the gel comprises 2.8% by weight citrus complex carbohydrate, 3.7% by weight cellulose derivative, 14.4% by weight polyol, 0.9% by weight ionic cross-linking agent and 78.2% by weight water.

In a further aspect of the invention, there is provided a process for making a gel, comprising the steps of: preparing a first aqueous solution comprising a citrus complex carbohydrate; preparing a second aqueous solution comprising a cellulose derivative; preparing a third aqueous solution comprising an ionic cross-linking agent; blending said first, second and third solutions to effect formation of ionic bonds between said citrus complex carbohydrate and said cellulose derivatives; and adding a polyol to the blended solutions to form a gel.

Embodiments of a gel and method of making a gel in accordance with the invention will now be described, by way of example only, with reference to the accompanying drawings, in which:

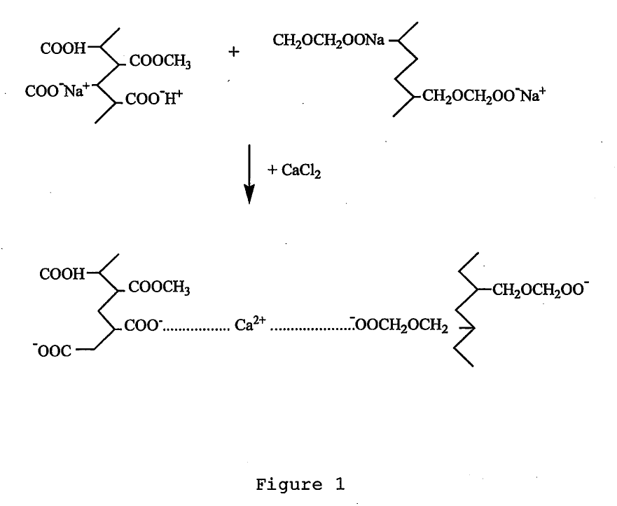

FIG. 1 illustrates the cross-linking of a citrus complex carbohydrate and a cellulose derivative in the presence of an ionic cross-linking agent, during gel formation.

The gel of the invention comprises a citrus complex carbohydrate, cross-linked with a cellulose derivative, wherein said cross-linking is facilitated by the presence of multivalent ions. As illustrated in the embodiment of FIG. 1, the cellulose derivative is sodium carboxymethyl cellulose and the multivalent ions are calcium ions, provided in the form of calcium chloride.

The gel is normally sterilised prior to use. The gel may be sterilised before being packaged and subsequently may be packaged aseptically. A preferred means of sterilisation is by electron beam (e-beam).

The gel of the invention can be applied to various wound types in order to promote healing. Examples of wounds include burns, eczema, nappy rash, cuts, grazes, ulcers and pressure sores. The gel can be applied alone, or in combination with a suitable wound dressing.

The gel can be applied using a syringe. Suitable syringes include those made of polycarbonate or polypropylene. The syringes are preferably transparent and display graduation markings on the surface thereof to allow accurate calculation of the amount of gel inserted into the wound.

Different sized syringes can be used. The plunger of the syringe can be assigned a different colour in order to denote the weight of the gel inside the syringe.

The invention is further illustrated by the following Example.

### EXAMPLE 1

A gel composition was prepared by mixing together the following components:

15 g citrus peel powder
20 g sodium carboxymethylcellulose
5 g calcium chloride
78 g polypropylene glycol
425 g water.

An aqueous blend of the citrus peel powder, manufactured by CP Kelco, and the sodium carboxymethylcellulose was prepared in 400 g water and an aqueous solution of the calcium chloride in 25 g water added thereto stepwise with mixing. The polypropylene glycol was then added to the blend and mixed to form a homogeneous gel. The gel was sterilised by electron beam (e-beam).

In simple trials application of the gel to a slight hot plate burn provided immediate cooling effect and pain relief within ten minutes. Application of the gel to nappy rash and baby eczema resulted in relief from all symptoms of both complaints within 48 hours.

### EXAMPLE 2

### Bacteriostatic Properties of the Gel Composition

The gel of the present invention can exhibit bacteriastatic properties. These bacteriastatic properties enable the gel to inhibit bacterial growth in appropriate conditions. The inhibition of bacterial growth in a wound which has been treated with the gel reduces infection and maldour in the wound.

TABLE 1
Showing Comparative Levels of Organism Growth

| | Organism Colony Count | | |
|---|---|---|---|
| | ORGANISM NAME | | |
| Product Type | S aureus | E coli | Ps aeruginosa |
| (1) A | 0 | 0 | 0 |
| | (after 7 days) | (after 7 days) | (after 7 days) |
| (1) B | 5.52 × 10⁴ | 1.93 × 10⁴ | 3.12 × 10³ |
| | | | (after 1 day) |
| (1) C | 7.66 × 10⁴ | 1.5 × 10³ | 0 |
| | | | (after 7 days) |

Table 1 illustrates the comparative level of organism growth of the gel of the present application, designated A, and two commercially available gels designated B and C respectively. The determination of bacteriostatic activity of each of the gels A to C was conducted using the SMTL Three Test Method.

After seven days the gel of the present invention showed no colony growth of staphylococcus aureus, Escherichia coli or Pseudomonas aeruginosa.

The commercially available gels B and C each showed significant growth of S. aureus and E. coli after seven and five days respectively. Gel B also showed significant P. aeruginosa growth after one day.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A gel for application to a wound, comprising a mixture of citrus complex carbohydrates, a cellulose derivative, a polyol component and water, wherein said citrus complex carbohydrates are cross-linked to said cellulose derivative by an ionic cross-linking agent.

2. A gel according to claim 1, wherein said cellulose derivative is a carboxymethyl cellulose derivative.

3. A gel according to claim 1, wherein said cellulose derivative is sodium carboxymethyl cellulose.

4. A gel according to claim 1, wherein said polyol component is a dihydroxyalkane having from 2 to 6 carbon atoms.

5. A gel according to claim 4, wherein said dihydroxyalkane is 1,2-dihydroxypropane.

6. A gel according to claim 1, wherein said polyol component is a polyhydroxyalkane of the general formula
CₙH₍₂ₙ₊₂₎Oₙ
wherein n is an integer of from 3 and 6.

7. A gel according to claim 6, wherein said polyol component is polyethylene glycol.

8. A gel according to claim 7, wherein said polyethylene glycol has a molecular weight in the range of from 200 to 600.

9. A gel according to claim 1, wherein said ionic cross-linking agent is a multivalent ion.

10. A gel according to claim 1, wherein said ionic cross-linking agent is a divalent ion.

11. A gel according to claim 10, wherein said divalent ion is a magnesium ion.

12. A gel according to claim 10, wherein said divalent ion is a calcium ion.

13. A gel according to claim 1, wherein said citrus complex carbohydrates have been extracted from citrus fruit peel by leaching using an aqueous medium.

14. A gel according to claim 13, wherein said aqueous medium is hot acidified water.

15. A gel according to claim 1, wherein said citrus complex carbohydrate is a low ester carbohydrate.

16. A gel according to claim 1, wherein said citrus complex carbohydrate comprises from 0.01 to 10% by weight of the gel.

17. A gel according to claim 16, wherein said citrus complex carbohydrate comprises about 2.8% by weight of the gel.

18. A gel according to claim 1, wherein said cellulose derivative comprises from 0.01% to 10% by weight of the gel.

19. A gel according to claim 18, wherein said cellulose derivative comprises about 3.7% by weight of the gel.

20. A gel according to claim 1, wherein said polyol component comprises from 0.1 to 30% by weight of the gel.

21. A gel according to claim 1, wherein said polyol comprises about 14.4% by weight of the gel.

22. A gel according to claim 1, wherein said ionic cross-linking agent comprises from 0.01 to 5% by weight of the gel.

23. A gel according to claim 1, wherein said ionic cross-linking agent comprises about 0.9% by weight of the gel.

24. A gel according to claim 1, wherein said gel has been sterilised.

25. A gel according to claim 1, wherein said gel further comprises an effective amount of at least one of the following components: an anti-bacterial agent; an anti-fungal agent; an anti-mycotic agent; an anaesthetic; an additional debriding agent; an anti-inflammatory agent; a growth factor; an enzyme; a pharmaceutical composition; vitamins; amino acids; aloe vera or trace metals.

26. A gel according to claim 1, wherein said gel comprises about 2.8% by weight citrus complex carbohydrate, about 3.7% by weight cellulose derivative, about 14.4% by weight polyol, about 0.9% by weight ionic cross-linking agent and about 78.2% by weight water.

27. A process for making a gel, comprising the steps of:
mixing a citrus complex carbohydrate, a cellulose derivative and an ionic cross-linking agent in aqueous solution to effect formation of ionic bonds between said citrus complex carbohydrate and said cellulose derivatives; and
adding a polyol to the mixture to form a gel.

28. A process according to claim 27, comprising the steps of:
preparing a first aqueous solution comprising a citrus complex carbohydrate;
preparing a second aqueous solution comprising a cellulose derivative;
preparing a third aqueous solution comprising an ionic cross-linking agent;
blending said first, second and third solutions to effect formation of ionic bonds between said citrus complex carbohydrate and said cellulose derivatives; and adding a polyol to the blended solutions to form a gel.

29. (canceled)

30. (canceled)

31. A wound dressing comprising a gel in accordance with claim 1.

32. A bacteriostatic gel comprising a mixture of citrus complex carbohydrates, a cellulose derivative, a polyol component and water, wherein said citrus complex carbohydrates are cross-linked to said cellulose derivative by an ionic cross-linking agent.

33. A gel according to claim 2, wherein said cellulose derivative is sodium carboxymethyl cellulose.

34. A gel according to claim 2, wherein said polyol component is a dihydroxyalkane having from 2 to 6 carbon atoms.

35. A gel according to claim 3, wherein said polyol component is a dihydroxyalkane having from 2 to 6 carbon atoms.

36. A gel according to claim 34, wherein said dihydroxyalkane is 1,2-dihydroxypropane.

37. A gel according to claim 35, wherein said dihydroxyalkane is 1,2-dihydroxypropane.

38. A gel according to claim 2, wherein said polyol component is a polyhydroxyalkane of the general formula
CₙH₍₂ₙ₊₂₎Oₙ
wherein n is an integer of from 3 and 6.

39. A gel according to claim 3, wherein said polyol component is a polyhydroxyalkane of the general formula
CₙH₍₂ₙ₊₂₎Oₙ
wherein n is an integer of from 3 and 6.

40. A gel according to claim 38, wherein said polyol component is polyethylene glycol.

41. A gel according to claim 39, wherein said polyol component is polyethylene glycol.

42. A gel according to claim 40, wherein said polyethylene glycol has a molecular weight in the range of from 200 to 600.

43. A gel according to claim 41, wherein said polyethylene glycol has a molecular weight in the range of from 200 to 600.
